(19) United States
(12) Patent Application Publication
(10) Pub. No.: US 2015/0098896 A1
(43) Pub. Date: Apr. 9, 2015

(54) SET AND METHOD FOR THE PRODUCTION OF A RADIOPHARMACEUTICAL

(71) Applicant: ZENTRALKLINIK BAD BERKA GMBH, Bad Berka (DE)

(72) Inventor: Dirk Mueller, Milda (DE)

(21) Appl. No.: 14/402,051

(22) PCT Filed: May 14, 2013
(86) PCT No.: PCT/EP2013/059896

§ 371 Date: Nov. 18, 2014

(30) Foreign Application Priority Data
May 18, 2012 (DE) 10 2012 208 378.2

(51) Int. Cl. A61K 51/08

(52) U.S. Cl. CPC A61K 51/081
USPC 424/1.11; 422/159

(57) ABSTRACT

A set for producing a radiopharmaceutical, having: a cation exchange cartridge; a reaction vial containing a precursor marker; a solution vial containing a solvent; an elution vial containing a sterile solution that includes sodium chloride (NaCl) and hydrochloric acid (HCl); a buffer salt. A method for producing a radiopharmaceutical is also disclosed.

The invention relates to a kit and to a method for producing a radiopharmaceutical.

Imaging techniques for medical diagnosis are commonplace, and in some cases have been so for decades. In some of these techniques, examples being positron emission spectroscopy (PET) or single photon emission computer tomography (SPECT), peptides, as for example edotreotide (DOTATOC), are labeled with radionuclides, as for example ⁶⁸gallium, and used as radiopharmaceuticals, also called tracers. Within the human body, the radiopharmaceutical binds to particular receptors, which especially in the case of tumor cells are overexpressed. By means of the imaging techniques, the elevated beta-plus decay of the ⁶⁹gallium can be ascertained and localized. According to [I. Velikyan: Synthesis, Characterisation and Application of ⁶⁸Ga-labelled Macromolecules. Dissertation, Uppsala University, 2005], the ⁶⁸gallium isotope decays with a half-life of 67.629 minutes to an extent of 89% with emission of a positron with at most 1.9 MeV, and to an extent of 11% with electron capture; the product in each case is the stable isotope ⁶⁸zinc. In nuclear medicine application, the positron which has been emitted collides with an electron after a few millimeters, with which it breaks down to form two photons each with 511 keV, the two photons being irradiated from the annihilation site at an angle of virtually 180° from one another. The irradiated photons can be detected with appropriate detectors, and the location of the annihilation can be determined very precisely by reconstruction of a plurality of detection events.

In view of the short half-life of ⁶⁸gallium, the radiopharmaceutical cannot be held for a prolonged time, but must instead be prepared a relatively short time prior to the intended use.

⁶⁸Gallium is generated by what are called gallium-68 generators, also called ⁶⁸Ge/⁶⁸Ga generators, from ⁶⁸germanium. ⁶⁸Germanium has a half-life of 270.8 days and decays into ⁶⁸gallium. This accumulates in the generator to a concentration governed by its own decay. The ⁶⁸gallium formed is separated from the stationary phase of the ⁶⁸germanium mother nuclide by means of a solvent which is introduced into the generator and with which only gallium, but not germanium, is eluted.

In known methods, hydrochloric acid with a normality of 0.05 N to 0.4 N is used for the eluting. The elution volume in this case is between 5 ml and 10 ml. The eluate, accordingly, contains hydrochloric acid and cannot be used directly to label peptides.

A variety of solutions have been disclosed for this problem.

In the case of the method of anionic concentration, the eluate is admixed with a large volume of concentrated hydrochloric acid, the ⁶⁸Ga is collected by means of an anion exchanger, and it is then eluted with water into a HEPES buffer solution (2-(4-(2-hydroxyethyl)-1-piperazinyl)ethanesulfonic acid) for the labeling of, for example, peptides. With this method, subsequent purification of the product is required, in other words the removal of unwanted substances. Moreover, large quantities of hydrochloric acid must be used.

Also known is combined cationic/anionic concentration, in which case two different cartridges are used for the cation exchange (SCX—strong cation exchanger) and for the anion exchange (SAX—strong anion exchanger).

With the cationic concentration method, the ⁶⁸gallium is held on a cation exchanger (SCX) and then eluted with an acetone/hydrochloric acid solution. The product obtained therefore comprises acetone, which, prior to use in the human body, must be removed by distillation at temperatures above 90° C. In order to verify complete removal of the acetone, intensive quality control is required, by means of a gas chromatograph, for example.

It is an object of the invention to specify a kit for the improved production of a radiopharmaceutical, and also to specify a corresponding improved method.

The object is achieved in accordance with the invention by a kit having the features of claim 1 and by a method having the features of claim 12.

Advantageous embodiments of the invention are subject matter of the dependent claims.

A kit of the invention comprises:
a sterile cation exchange cartridge (SCX cartridge),
a reaction vial with a labeling precursor, more particularly a lyophilized labeling precursor,
a solution vial with a solvent, such as a sterile aqueous solution of acetic acid and hydrochloric acid,
an elution vial with sterile sodium chloride/hydrochloric acid solution,
a buffer salt.

A vial may also be termed an ampoule or septum bottle.

The buffer salt may be present, for example, in the reaction vial or in the solution vial.

The contents of the reaction vial have preferably been lyophilized.

Additionally provided in the reaction vial may be lyophilized ascorbic acid or another suitable stabilizer. The stabilizer prevents radiolytic degradation of the labeled substance during the use of the radiopharmaceutical.

As buffer salt, for example, ammonium acetate or sodium acetate may be used.

The kit is used as follows:

A ⁶⁸Ge/⁶⁸Ga generator provides the ⁶⁸gallium needed for labeling. The ⁶⁸Ge/⁶⁸Ga generator is eluted using hydrochloric acid, with a concentration of 0.1 mol/l, for example. In this way, 68gallium is eluted. The generator eluate is supplied to the SCX cartridge. The SCX cartridge used may be, for example, a silica gel-based (silica based) cartridge. The SCX cartridge is preconditioned, for example, with 1 ml of hydrochloric acid of 5.5 mol/l concentration, and 10 ml of water. The preferably lyophilized mixture in the reaction vial is dissolved with the solvent from the solution vial. The SCX cartridge is then eluted, using the solution from the elution vial, into the reaction vial.

The reaction solution which is produced in the reaction vial may optionally be heated at 90° C. to 100° C., over a time of 5 minutes to 15 minutes, for example, more particularly seven minutes, in order to accelerate the reaction, in which the ⁶⁸gallium joins with the labeling precursor to form the tracer. The reaction may also take place at room temperature, in which case a correspondingly greater amount of time may be needed.

The concentration of unbound ^{H}gallium is preferably smaller than 5%. The radiochemical purity of the tracer is greater than 95%. The reaction mixture contains no toxic or objectionable substances, and so there is no need for subsequent purification. After sterile filtration, carried out optionally, the radiochemical yield is around 82% (n.d.c.—non decay corrected).

At the end of the reaction, the radiopharmaceutical may be neutralized by addition of a sterile phosphate buffer, an example being 2 ml of sodium phosphate, 1 mmol/ml Na⁺, 0.6 mmol/ml PO₄³⁻, pH 7.0.

Quality control by thin-layer chromatography may then follow. The tracer thus produced can be used subsequently, without further purification, as a radiopharmaceutical.

The kit of the invention can be used for routinely available application in clinical practice in the context of ⁶⁸Ga labeling procedures. The kit of the invention reduces the level of operation with concentrated hydrochloric acid during the purifying and concentrating procedure on the ⁶⁸Ga eluate. The attainable end product (tracer) is available with high purity and in a high yield of around 80% to 95%. As a result, it is likewise possible to avoid the use of acetone or other organic solvents or compounds such as 2-(4-(2-hydroxyethyl)-1-piperazinyl)ethanesulfonic acid (HEPES). In this way, there is also no need, relative to methods known from the prior art, for verification that the acetone has been removed completely, and so there is no requirement for intensive quality control, by means of a gas chromatograph, for example. In this way, it is made possible to produce kits which can be employed by medical staff in a relatively simple way, by adding the solution to the lyophilized mixture, without any need for costly and complicated laboratory equipment.

The tracers obtained are stable for longer than tracers known from the prior art, allowing multi-dose products to be produced for the labeling and investigation of a number of patients.

In one embodiment of the invention, the reaction vial contains a lyophilized mixture of sodium acetate and macroaggregated Human Serum Albumin (HSA). The tracer thus formed can be used in particular for perfusion diagnosis by means of positron emission tomography.

Instead of sodium acetate, ammonium acetate may be used in principle, but sodium acetate is more suitable for lyophilization.

In one embodiment of the invention, the reaction vial contains:
at most 20 mg, preferably at most 2 mg, of HSA,
22 mg to 40 mg, preferably 27.6 mg, of buffer salt, more particularly sodium acetate,
at most 100 mg, preferably at most 5 mg, of L-ascorbic acid.

In one embodiment of the invention, the solution vial contains:
1 ml to 10 ml of water and also hydrochloric acid and acetic acid in an amount such that the pH of the solution composed of the contents of the reaction vial, the solvent from the solution vial, and the elution vial solution used to elute the SCX cartridge is between 3 and 4.

In one embodiment of the invention, the solution vial contains:
1 ml to 10 ml, preferably 1 ml to 7 ml, of water
4 μl to 10 μl, preferably 6.73 μl, of concentrated hydrochloric acid
4 μl to 10 μl, preferably 6 μl to 8 μl, of acetic acid.

In one embodiment of the invention, the elution vial contains 0.25 ml to 3 ml of elution solution composed of 5 mol/l sodium chloride and 5.5 mol/l hydrochloric acid with 12 μl to 100 μl, preferably 25 μl, of 5.5 mol/l hydrochloric acid per ml of 5 mol/l sodium chloride. The SCX cartridge is preferably eluted with 0.5 ml of the NaCl/HCl elution solution.

A method of the invention for producing a radiopharmaceutical comprises the following steps:
obtaining a generator eluate comprising ⁶⁸gallium from a ⁶⁸Ge/⁶⁸Ga generator by means of hydrochloric acid,
passing the generator eluate into a cation exchange cartridge in which the ^{H}gallium is held,
removing an effluent of the generator eluate from the cation exchange cartridge,
eluting the ⁶⁸gallium from the cation exchange cartridge by means of a solution comprising sodium chloride and hydrochloric acid and passing it into a mixture of a labeling precursor and sodium acetate.

In one embodiment, the method may be carried out by means of the kit of the invention.

Working examples of the invention are elucidated in more detail below with reference to drawings.

In these drawings:

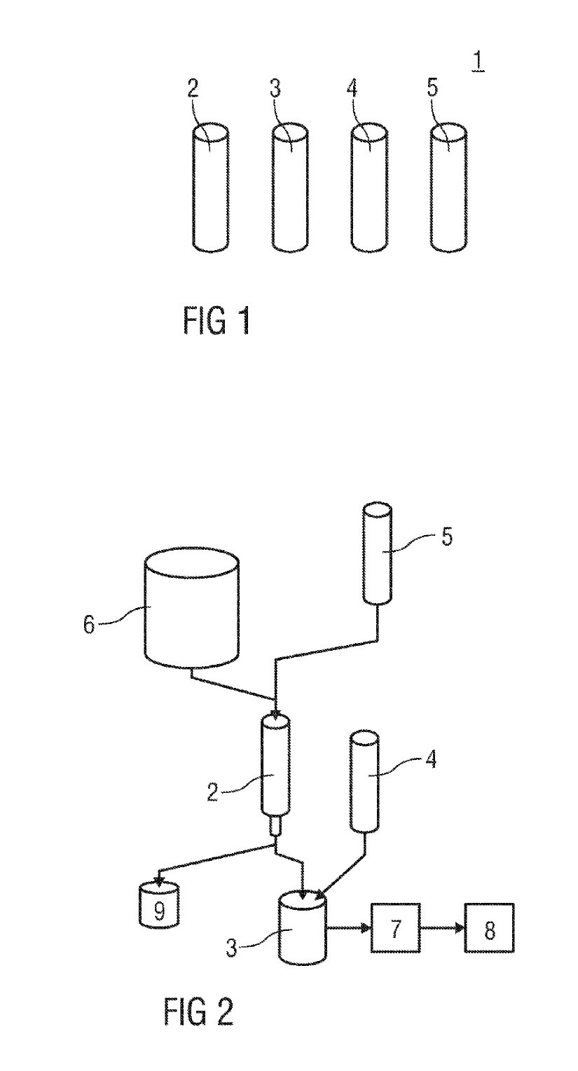

FIG. 1 shows a schematic view of a kit for producing a radiopharmaceutical, and

FIG. 2 shows an arrangement for producing a radiopharmaceutical by means of the kit.

Parts corresponding to one another bear the same reference numerals in all the figures.

FIG. 1 shows a schematic view of a kit 1 for producing a radiopharmaceutical. The kit 1 comprises:
a cation exchange cartridge 2,
a reaction vial 3 with a mixture comprising a labeling precursor and a buffer salt,
a solution vial 4 with a solvent,
an elution vial 5 with a sterile solution comprising sodium chloride NaCl and hydrochloric acid HCl.

The labeling precursor present in the reaction vial 3 is macroaggregated Human Serum Albumin HSA.

The mixture in the reaction vial 3 has been lyophilized.

The mixture in the reaction vial 3 optionally comprises ascorbic acid C₆H₈O₆ or another radical scavenger.

The solvent is preferably formed as an aqueous solution from acetic acid C₂H₄O₂ and hydrochloric acid HCl.

As the buffer salt, ammonium acetate CH₃COONH₄ or sodium acetate C₂H₃NaO₂ is provided.

The cation exchange cartridge 2 may be preconditioned with hydrochloric acid HCl and water H₂O, in particular with 1 ml of hydrochloric acid HCl of concentration 5.5 mol/l and 10 ml of water H₂O.

The reaction vial 3 contains:
at most 20 mg, preferably at most 2 mg, of Human Serum Albumin HSA,
22 mg to 40 mg, preferably 27.6 mg, of buffer salt, more particularly sodium acetate C₂H₃NaO₂,
at most 100 mg, preferably at most 5 mg, of L-ascorbic acid C₆H₈O₆.

The solution vial 4 contains:
1 ml to 10 ml, preferably 1 ml to 7 ml, of water H₂O
4 μl to 10 μl, preferably 6.73 μl, of concentrated hydrochloric acid HCl
4 μl to 10 μl, preferably 6 μl to 8 μl, of acetic acid C₂H₄O₂.

The elution vial 5 contains an amount of 0.25 ml to 3 ml of elution solution composed of 5 mol/l sodium chloride NaCl and 5.5 mol/l hydrochloric acid HCl with 12 μl to 100 μl, preferably 25 μl, of 5.5 mol/l hydrochloric acid HCl per ml of 5 mol/l sodium chloride NaCl.

The kit 1 may additionally comprise a vial with a neutralizing buffer, more particularly a sodium phosphate buffer.

FIG. 2 shows an arrangement for producing a radiopharmaceutical 8 by means of the kit 1.

A ⁶⁸Ge/⁶⁸Ga generator 6 provides the ^{H}gallium needed for labeling. The ⁶⁸Ge/⁶⁸Ga generator 6 is eluted using hydrochloric acid HCl, with a concentration of 0.1 mol/l, for example. In this way, ⁶⁸gallium is eluted and is held on the cation exchange cartridge 2. The generator eluate is supplied to the cation exchange cartridge 2. The 0.1 mol/l HCl effluent, possibly with traces of the ⁶⁸germanium mother nuclide, is collected separately in a waste collecting vessel 9, and disposed of in line with the statutory provisions. The lyophilized mixture in the reaction vial 3 is dissolved with the solvent from the solution vial 4. The cation exchange cartridge 2 is then eluted by means of the solution from the elution vial 5 into the reaction vial 3.

The reaction solution which is produced in the reaction vial 3 may optionally be heated at 90° C. to 100° C., over a time of 5 minutes to 15 minutes, for example, more particularly seven minutes, in order to accelerate the reaction, in which the ⁶⁸gallium joins with the labeling precursor to form the radiopharmaceutical 8, also called tracer. The reaction may also take place at room temperature, in which case it requires a correspondingly greater amount of time.

At the end of the reaction, a sterile phosphate buffer may be added.

The tracer thus produced can then be used as radiopharmaceutical 8.

### LIST OF REFERENCE NUMERALS

1 Kit

2 Cation exchange cartridge

3 Reaction vial

4 Solution vial

5 Elution vial

⁶⁸Ge/⁶⁸Ga generator

8 Radiopharmaceutical

9 Waste collecting vessel

1-12. (canceled)

13. A kit for producing a radiopharmaceutical, comprising:
a cation exchange cartridge,
a reaction vial with macroaggregated Human Serum Albumin (HSA) as labeling precursor,
a solution vial with a solvent formed from an aqueous solution of acetic acid (C₂H₄O₂) and hydrochloric acid (HCl),
an elution vial with a sterile solution comprising sodium chloride (NaCl) and hydrochloric acid (HCl), and
a buffer salt.

14. The kit as claimed in claim 13, wherein the buffer salt is present in the reaction vial or in the solution vial.

15. The kit as claimed in claim 13, wherein the mixture in the reaction vial has been lyophilized.

16. The kit as claimed in claim 13, wherein the mixture in the reaction vial comprises ascorbic acid (C₆H₈O₆).

17. The kit as claimed in claim 13, wherein ammonium acetate (CH₃COONH₄) or sodium acetate (C₂H₃NaO₂) is provided as buffer salt.

18. The kit as claimed in claim 13, wherein the reaction vial contains:
at most 20 mg, preferably at most 2 mg, of Human Serum Albumin (HSA),
22 mg to 40 mg, preferably 27.6 mg, of buffer salt, more particularly sodium acetate (C₃H₃NaO₂), and
at most 100 mg, preferably at most 5 mg, of L-ascorbic acid (C₆H₈O₆).

19. The kit as claimed in claim 13, wherein the solution vial contains:
ml to 10 ml, preferably 1 ml to 7 ml, of water (H₂O)
4 μl to 10 μl, preferably 6.73 μl, of concentrated hydrochloric acid (HCl)
4 μl to 10 μl, preferably 6 μl to 8 μl, of acetic acid (C₂H₄O₂).

20. The kit as claimed in claim 13, wherein the elution vial contains 0.25 ml to 3 ml of elution solution composed of 5 mol/l sodium chloride (NaCl) and 5.5 mol/l hydrochloric acid (HCl) with 12 μl to 100 μl, preferably 25 μl, of 5.5 mol/l hydrochloric acid (HCl) per ml of 5 mol/l sodium chloride (NaCl).

21. The kit as claimed in claim 13, further comprising a vial with a neutralizing buffer, more particularly a sodium phosphate buffer.

22. A method for producing a radiopharmaceutical, comprising the following steps:
obtaining a generator eluate comprising ⁶⁸gallium from a ⁶⁸Ge/⁶⁸Ga generator by means of hydrochloric acid (HCl),
passing the generator eluate into a cation exchange cartridge in which the ⁶⁸gallium is held,
removing an effluent of the generator eluate from the cation exchange cartridge, and
eluting the ⁶⁸gallium from the cation exchange cartridge by means of a solution comprising sodium chloride (NaCl) and hydrochloric acid (HCl) and passing it into a mixture of macroaggregated Human Serum Albumin (HSA) as labeling precursor and sodium acetate (C₂H₃NaO₂).
